(19) United States
(12) Patent Application Publication
(10) Pub. No.: US 2026/0041650 A1
(43) Pub. Date: Feb. 12, 2026

(54) COMPOSITION FOR LIVING BODY

(71) Applicant: FUJIFILM Corporation, Tokyo (JP)

(72) Inventors: Haruki TOMIKAWA, Kanagawa (JP); Toshihide Yoshitani, Kanagawa (JP)

(73) Assignee: FUJIFILM Corporation, Tokyo (JP)

(21) Appl. No.: 19/361,038

(22) Filed: Oct. 17, 2025

Related U.S. Application Data
(63) Continuation of application No. PCT/JP2024/014935, filed on Apr. 15, 2024.

(30) Foreign Application Priority Data
Apr. 21, 2023 (JP) 2023-069865

(51) Int. Cl. A61K 31/08; A61K 31/683; A61K 47/10

(52) U.S. Cl. CPC A61K 31/08; A61K 31/683; A61K 47/10

(57) ABSTRACT

An object of the present invention is to provide a composition for a living body, which forms a film upon contact with water, the formed film having excellent elastic modulus and strain resistance. The composition for a living body according to the embodiment of the present invention contains a compound X selected from the group consisting of an α-monoalkyl glyceryl ether and an α-monoalkenyl glyceryl ether, a phospholipid, and a compound Y selected from the group consisting of an alcohol having 4 or less carbon atoms and a polyalkylene oxide, in which a content of the compound Y is 1% to 10% by mass with respect to a total mass of the compound X and the phospholipid.

### CROSS-REFERENCE TO RELATED APPLICATIONS

This application is a Continuation of PCT International Application No. PCT/JP2024/014935 filed on Apr. 15, 2024, which claims priority under 35 U.S.C. § 119 (a) to Japanese Patent Application No. 2023-069865 filed on Apr. 21, 2023. The above applications are hereby expressly incorporated by reference, in their entirety, into the present application.

### BACKGROUND OF THE INVENTION

### 1. Field of the Invention

The present invention relates to a composition for a living body.

### 2. Description of the Related Art

Conventionally, there is a demand for suppressing pain caused by inflammation or the like in a mucous membrane or a skin.

For example, in cancer patients, cancer treatment affects the oral mucosa, making stomatitis more likely to occur. In anti-cancer drug treatment, stomatitis inevitably occurs in a case where a drug that easily cause stomatitis is administered, in radiation therapy for head and neck cancer (cancer in the region from the head to the neck), stomatitis is inevitable when radiation is directly applied to the oral mucosa. The pain of stomatitis is so strong that it is difficult to eat a meal by mouth.

Examples of a symptomatic treatment for stomatitis include a patch (for example, Aphthaseal® 25 μg, manufactured by Taisho Pharmaceutical Co., Ltd., active ingredient: triamcinolone acetonide) that is directly attached to the affected part, an ointment (for example, Dexaltin oral ointment, manufactured by Nippon Kayaku Co., Ltd., active ingredient: dexamethasone) that is applied to the affected part, and a spray agent (for example, Salcoat® capsule for topical use 50 μg, manufactured by TEIJIN PHARMA LIMITED, active ingredient: beclomethasone propionate ester) that is sprayed on the affected part, and the like.

However, when eating a meal by mouth, the patch attached to the affected part may be peeled off, or the ointment or the spray agent applied to the affected part may be lost, and thus the pain of stomatitis cannot be suppressed sufficiently.

As a composition that can exhibit an anti-inflammatory effect and the like, JP2010-059066A discloses an alkylglyceryl ether-formulated composition containing (A) alkylglyceryl ether which is one or two or more selected from the group consisting of chimyl alcohol, batyl alcohol, and selachyl alcohol, (B) lecithin, (C) nonionic surfactant, and (D) polyhydric alcohol, in which (A)/(B)=10/1 to 10/10, [(A)+ (B)]/(C)=10/1 to 10/10, [(A)+ (B)]/(D)=10/5 to 10/100, and a blending amount of (A) in the composition is 5% to 50% by mass.

### SUMMARY OF THE INVENTION

A material used for a living body is often used in an environment in contact with water in the atmosphere and an aqueous fluid containing water such as body fluid, and such a material is required to be less likely to be removed from a biological surface under the above-described environment as described above.

To exhibit such performance, specifically, it is necessary that the elastic modulus and the strain resistance of the film formed by bringing the composition for a living body into contact with water are excellent.

As a result of studying the composition according to JP2010-059066A, the inventors of the present invention have found that the elastic modulus and strain resistance of the above-described film do not satisfy the desired level, and further improvement is required.

Therefore, an object of the present invention is to provide a composition for a living body, which forms a film upon contact with water, the formed film having excellent elastic modulus and strain resistance.

As a result of conducting an extensive investigation to achieve the objects, the present inventors have found that the objects can be achieved by the following constitution.

[1] A composition for a living body, comprising: a compound X selected from the group consisting of an α-monoalkyl glyceryl ether and an α-monoalkenyl glyceryl ether; a phospholipid; and a compound Y selected from the group consisting of an alcohol having 4 or less carbon atoms and a polyalkylene oxide, in which a content of the compound Y is 1% to 10% by mass with respect to a total mass of the compound X and the phospholipid.

[2] The composition for a living body according to [1], in which the compound X is selected from the group consisting of selachyl alcohol, batyl alcohol, and chimyl alcohol.

[3] The composition for a living body according to [1] or [2], in which the compound X is selachyl alcohol.

[4] The composition for a living body according to any one of [1] to [3], in which a mass ratio of a content of the compound X to a content of the phospholipid is 70/30 to 50/50.

[5] The composition for a living body according to any one of [1] to [4], in which a content of water is 0% to 10% by mass with respect to a total mass of the composition for a living body.

[6] The composition for a living body according to any one of [1] to [5], in which a content of the compound X is 50% by mass or more with respect to a total mass of the composition for a living body.

[7] The composition for a living body according to any one of [1] to [6], in which the composition for a living body forms an inverse hexagonal columnar phase through absorption of water or moisture.

[8] The composition for a living body according to any one of [1] to [7], in which the composition for a living body is for a skin or a mucous membrane.

According to the present invention, it is possible to provide a composition for a living body, which forms a film upon contact with water, the formed film having excellent elastic modulus and strain resistance.

### DESCRIPTION OF THE PREFERRED EMBODIMENTS

Hereinafter, the present invention will be described in detail.

The description of the configuration requirements described below is made on the basis of representative embodiments of the present invention, but it should not be construed that the present invention is limited to those embodiments.

In the present specification, any numerical range expressed by using “to” means a range including the numerical values before and after the “to” as a lower limit value and an upper limit value, respectively.

In addition, in the present specification, in a case where there are two or more components corresponding to a certain component, “content” of such a component means the total content of the two or more components.

In a range of numerical values described in stages in the present specification, the upper limit value or the lower limit value described in a certain range of numerical values may be replaced with an upper limit value or a lower limit value of the range of numerical values described in other stages. In addition, regarding the numerical range described in the present specification, an upper limit value or a lower limit value described in a numerical value may be replaced with a value described in Examples.

In the present specification, a combination of two or more preferred embodiments is a more preferred embodiment.

### [Composition for Living Body]

Hereinafter, the composition for a living body according to the embodiment of the present invention will be described in detail.

The composition for a living body according to the embodiment of the present invention contains a compound X selected from the group consisting of an α-monoalkyl glyceryl ether and an α-monoalkenyl glyceryl ether, a phospholipid, and a compound Y selected from the group consisting of an alcohol having 4 or less carbon atoms and a polyalkylene oxide, in which a content of the compound Y is 1% to 10% by mass with respect to a total mass of the compound X and the phospholipid.

Although the reason why the composition for a living body having the above-mentioned configuration can achieve the objects of the present invention is not always clear, the present inventors speculate as follows.

The mechanism by which the effect is obtained is not limited by the following supposition. In other words, even in a case where an effect is obtained by a mechanism other than the following, it is included in the scope of the present invention.

The composition for a living body according to the embodiment of the present invention contains a compound X selected from the group consisting of an α-monoalkyl glyceryl ether and an α-monoalkenyl glyceryl ether, a phospholipid, and a compound Y selected from the group consisting of an alcohol having 4 or less carbon atoms and a polyalkylene oxide, which can form a film (preferably a film exhibiting a liquid crystal phase) having excellent biological adhesiveness and strength (for example, an elastic modulus and strain resistance) upon contact with water. In particular, it is presumed that the composition for a living body according to the embodiment of the present invention contains the above-described components at a specific content ratio, whereby the elastic modulus and the strain resistance of the formed film are excellent.

Hereinafter, the fact that at least one of the elastic modulus or the strain resistance of the film formed upon contact with water is more excellent is also referred to as “the effect of the present invention is more excellent”.

Hereinafter, each component that can be contained in the composition for a living body according to the embodiment of the present invention will be described in detail.

[Compound X Selected from Group Consisting of α-Monoalkyl Glyceryl Ether and α-monoalkenyl Glyceryl Ether]

The composition for a living body according to the embodiment of the present invention contains a compound X selected from the group consisting of α-monoalkyl glyceryl ether and α-monoalkenyl glyceryl ether.

The number of carbon atoms in the alkyl group of the α-monoalkyl glyceryl ether is preferably 6 to 32, more preferably 10 to 24, and still more preferably 16 to 18.

The number of carbon atoms in the alkenyl group of the α-monoalkenyl glyceryl ether is preferably 6 to 32, more preferably 10 to 24, and still more preferably 16 to 18.

The number of double bonds in the alkenyl group is not particularly limited as long as it is 1 or more, but is preferably 1 to 3 and more preferably 1.

The alkyl group and the alkenyl group may be linear or branched, but are preferably linear.

Examples of the alkyl group and the alkenyl group include an oleyl group, a stearyl group, a cetyl group, a lauryl group, a tridecyl group, a myristyl group, a pentadecyl group, a mono-iso-stearyl group, and the like.

The compound X is also preferably α-monoalkenyl glyceryl ether.

Examples of the compound X include selachyl alcohol (α-mono-oleyl glyceryl ether), batyl alcohol (α-monostearyl glyceryl ether), chimyl alcohol (α-monocetyl glyceryl ether), α-monolauryl glyceryl ether, α-monotridecyl glyceryl ether, α-monomyristyl glyceryl ether, α-monopentadecyl glyceryl ether, α-mono-isostearyl glyceryl ether, and the like, and selachyl alcohol, batyl alcohol, or chimyl alcohol is preferable and selachyl alcohol is more preferable.

Among these, the compound X is preferably selected from the group consisting of selachyl alcohol, batyl alcohol, and chimyl alcohol, and more preferably selachyl alcohol.

The compound X may be used alone or in combination of two or more types thereof. As the compound X, α-monoalkyl glyceryl ether and α-monoalkenyl glyceryl ether may be used in combination.

From the viewpoint that the strain resistance and the liquid viscosity are more excellent, the content of the compound X is preferably 30% by mass or more, more preferably 45% by mass or more, and still more preferably 50% by mass or more with respect to the total mass of the composition for a living body. In addition, from the viewpoint that the elastic modulus, the strain resistance, and the film formation rate are more excellent, the content of the compound X is preferably 90% by mass or less, more preferably 80% by mass or less, still more preferably 70% by mass or less, and particularly preferably 65% by mass or less with respect to the total mass of the composition for a living body.

The content of the compound X means the total content of α-monoalkyl glyceryl ether and α-monoalkenyl glyceryl ether contained in the composition for a living body.

The liquid viscosity is a viscosity of the composition for a living body before being brought into contact with water, and the liquid viscosity is preferably low from the viewpoint that the composition is easy to apply and has an excellent feeling of use.

It is also preferable that the composition for a living body according to the embodiment of the present invention has a high film formation rate in use. The above-described film formation rate may also correspond to a formation rate of a liquid crystal film (a film containing a liquid crystal phase), that is, a formation rate of a liquid crystal phase.

### [Phospholipid]

The composition for a living body according to the embodiment of the present invention contains a phospholipid.

The phospholipid is not particularly limited as long as it has a phosphate ester structure in the molecular structure thereof; however, a glycerophospholipid having glycerin as a skeleton and a sphingophospholipid having sphingosine as a skeleton are typical. Both the glycerol phospholipid and the sphingolipid have, in the molecule, an acyl group derived from a fatty acid.

The number of carbon atoms in the acyl group contained in the phospholipid is not particularly limited, but is preferably 12 to 22 and more preferably 16 to 18.

The hydrocarbon group as the above-described acyl group, excluding a carbonyl group, is preferably a saturated or unsaturated chain hydrocarbon group having 11 to 21 carbon atoms, and more preferably a saturated or unsaturated chain hydrocarbon group having 15 to 17 carbon atoms. Specific examples of the above-described hydrocarbon group include CH₃(CH₂)₁₄—, CH₃(CH₂)₇CH═CH(CH₂)₇—, CH₃(CH₂)₄(CH═CHCH₂)₂(CH₂)₆—, and the like; however, the examples are not limited thereto.

In a case where the phospholipid has two or more acyl groups in the molecule, the acyl groups may be the same as or different from each other.

Examples of the phospholipid include phosphatidylcholine, lysophosphatidylcholine, phosphatidylethanolamine, phosphatidylinositol, phosphatidic acid, phosphatidylglycerol, sphingomyelin, sphingophosphoethanolamine, and the like.

From the viewpoints of improving the water absorption rate of the composition for a living body according to the embodiment of the present invention and easily forming a columnar phase having a large domain size, the phospholipid preferably includes an ionic phospholipid. It is noted that the water absorption rate means a rate at which the composition for a living body according to the embodiment of the present invention absorbs water or moisture. From the viewpoint that the film formation rate and the effects of the present invention are more excellent, it is preferable that the water absorption rate be high.

Examples of the ionic phospholipid include a phospholipid having a cationic moiety and an anionic moiety in one molecule, and specific examples thereof include phosphatidylcholine. Phosphatidylcholine is a phospholipid having N derived from choline as a cationic moiety and P—O⁻ derived from phosphoric acid as an anionic moiety.

The acyl group of the phosphatidylcholine is preferably an acyl group derived from palmitic acid (CH₃(CH₂)₁₄COOH), oleic acid (CH₃(CH₂)₇CH═CH(CH₂)₇COOH), or linoleic acid (CH₃(CH₂)₄(CH═CHCH₂)₂(CH₂)₆COOH).

Specific examples of the phosphatidylcholine include a PO phosphatidylcholine (a phosphatidylcholine having an acyl group derived from palmitic acid at the first position (α-position), an acyl group derived from oleic acid at the second position (β-position), and choline at the third position (γ-position)), a DL phosphatidylcholine (a phosphatidylcholine having an acyl group derived from linoleic acid at the first position (α-position), an acyl group derived from linoleic acid at the second position (β-position), and choline at the third position (γ-position)), and a dipalmitoyl phosphatidylcholine.

The phospholipid preferably includes phosphatidylcholine.

In a case where the phospholipid includes phosphatidylcholine, the content of the phosphatidylcholine is preferably 50% by mass or more, more preferably 75% by mass or more, and still more preferably 90% by mass or more with respect to the total mass of the phospholipid. The upper limit of the content of the phosphatidylcholine with respect to the total mass of the phospholipid is not particularly limited, and may be 100% by mass, and is generally 99% by mass or less.

It is noted that in the preparation of the composition for a living body, a composition containing a phospholipid may be used, and specifically, for example, a composition containing phosphatidylcholine may be used. In the composition containing the phosphatidylcholine, the content of the phosphatidylcholine is preferably 75% by mass or more and more preferably 90% by mass or more with respect to the total mass of the composition containing the phosphatidylcholine.

The phospholipid may be used alone or in combination of two or more types thereof.

From the viewpoint that the effect of the present invention is more excellent, the mass ratio (compound X/phospholipid) of the content of the compound X to the content of the phospholipid is preferably 95/5 to 20/80, more preferably 80/20 to 40/60, still more preferably 70/30 to 50/50, and particularly preferably 65/35 to 55/45.

From the viewpoint that the effect of the present invention is more excellent, the content of the phospholipid is preferably 5% to 80% by mass, more preferably 20% to 50% by mass, and still more preferably 30% to 45% by mass with respect to the total mass of the composition for a living body.

[Compound Y Selected from Group Consisting of Alcohol Having 4 or Less Carbon Atoms and Polyalkylene Oxide]

The composition for a living body according to the embodiment of the present invention contains a compound Y selected from the group consisting of an alcohol having 4 or less carbon atoms and a polyalkylene oxide.

The alcohol having 4 or less carbon atoms and the polyalkylene oxide can function as a solvent for the compound X and the phospholipid.

The alcohol having 4 or less carbon atoms and the polyalkylene oxide are preferably biocompatible.

### <Alcohol Having 4 or Less Carbon Atoms>

The alcohol having 4 or less carbon atoms is a compound in which at least one hydroxy group is bonded to an aliphatic hydrocarbon group having 4 or less carbon atoms. In a case where the alcohol has 5 or more carbon atoms, the function as a solvent for the compound X and the phospholipid is reduced and the solubility is insufficient, which is not preferable.

The aliphatic hydrocarbon group having 4 or less carbon atoms may be linear, branched, or cyclic, but is preferably linear or branched.

The number of carbon atoms in the alcohol having 4 or less carbon atoms is not particularly limited as long as it is 4 or less, but is preferably 3 or less. The lower limit of the number of carbon atoms in the alcohol having 4 or less carbon atoms is 1 or more, preferably 2 or more, and more preferably 3 or more.

The number of hydroxy groups in the alcohol having 4 or less carbon atoms is not particularly limited as long as it is 1 or more, but is preferably 1 or 2.

Examples of the alcohol having 4 or less carbon atoms include monoalcohols such as ethanol, propanol, isopropanol, and butanol, glycols (diols) such as ethylene glycol, propylene glycol, butylene glycol, and 1,3-butylene glycol, and glycerol, and ethanol, isopropanol, ethylene glycol, 1,3-butylene glycol, or propylene glycol is preferable, and ethanol, 1,3-butylene glycol, or propylene glycol is more preferable.

### <Polyalkylene Oxide>

The polyalkylene oxide is a polymer having an oxyalkylene group as a repeating unit. The terminus of the polyalkylene oxide may be any of an alkyl group or a hydroxy group.

The polyalkylene oxide may have a hydroxy group, and the number of hydroxy groups is not particularly limited.

The number of carbon atoms in the alkylene group contained in the polyalkylene oxide is not particularly limited, but is preferably 1 to 4. Examples of the oxyalkylene group include an oxyethylene group, an oxypropylene group, and the like.

The polyalkylene oxide may be linear or branched.

A plurality of oxyalkylene groups contained in the polyalkylene oxide may be the same as or different from each other. For example, the polyalkylene oxide may be a polymer having both an oxyethylene group and an oxypropylene group as repeating units.

Examples of the polyalkylene oxide include polyoxyethylene polyol, polyoxypropylene polyol, and polyoxyethylene polyoxypropylene polyol.

The compound Y may be used alone, or two or more types thereof may be used in combination. As the compound Y, an alcohol having 4 or less carbon atoms and a polyalkylene oxide may be used in combination.

The content of the compound Y is 1% to 10% by mass with respect to the total mass of the compound X and the phospholipid.

In a case where the content of the compound Y is less than 1% by mass with respect to the total mass of the compound X and the phospholipid, it is not preferable from the viewpoint that the composition cannot be homogenized and the film cannot be formed, and in a case where the content of the compound Y is more than 10% by mass, it is not preferable from the viewpoint that the strain resistance deteriorates.

The content of the compound Y means the total content of the alcohol having 4 or less carbon atoms and the polyalkylene oxide, which are contained in the composition for a living body. Therefore, in a case where the composition for a living body contains an alcohol having 4 or less carbon atoms and a polyalkylene oxide, the total content of the alcohol and the polyalkylene oxide satisfies the above-described requirement.

From the viewpoint of further improving the strain resistance and the film formation rate, the content of the compound Y is preferably 3% to 8% by mass with respect to the total mass of the compound X and the phospholipid.

The content of the compound Y is preferably 1% to 9% by mass and more preferably 3% to 8% by mass with respect to the total mass of the composition for a living body.

### [Water]

The composition for a living body according to the embodiment of the present invention may contain water.

In a case where the composition for a living body according to the embodiment of the present invention is used on the skin or the mucous membrane, from the viewpoint that the composition for a living body is not easily removed, the content of water is preferably 0% to 10% by mass, more preferably 0% to 5% by mass, still more preferably 0% to 1% by mass, and particularly preferably 0% by mass, with respect to the total mass of the composition for a living body.

### [Quaternary Ammonium Salt]

The composition for a living body according to the embodiment of the present invention may further contain a quaternary ammonium salt (excluding phosphatidylcholine).

The quaternary ammonium salt is preferably an ionic compound consisting of a polyatomic ion (quaternary ammonium cation) having a positive charge, which is represented by a molecular formula NR₄⁺, and an anion.

R's each independently represent an alkyl group which may have a substituent, an alkenyl group which may have a substituent, or an aryl group, and a plurality of R's may be the same or different from each other.

Examples of the substituent which may be contained in the alkyl group and the alkenyl group include a hydroxyl group, an acyloxy group, an acyl group, an alkoxy group, an alkenyloxy group, and an aryl group. The hydrocarbon group contained in the acyloxy group and the acyl group is preferably a long-chain alkyl group (an alkyl group having 8 or more carbon atoms) or a long-chain alkenyl group (an alkenyl group having 8 or more carbon atoms).

The anion is not particularly limited, and examples thereof include an acid-derived anion, a halide ion, a hydroxide ion, and the like.

As the quaternary ammonium salt, a di-long-chain alkyl quaternary ammonium salt or a di-long-chain alkenyl quaternary ammonium salt is preferable.

The di-long-chain alkyl quaternary ammonium salt and the di-long-chain alkenyl quaternary ammonium salt are salts in which two of R's in a quaternary ammonium cation represented by the molecular formula NR₄⁺ are a long-chain alkyl group (an alkyl group having 8 or more carbon atoms) or a long-chain alkenyl group (an alkenyl group having 8 or more carbon atoms). The other two R's are each independently a short-chain alkyl group (an alkyl group having 1 to 7 carbon atoms) or an aryl group.

The number of carbon atoms in the long-chain alkyl group and the long-chain alkenyl group is preferably 12 to 22 and more preferably 16 to 18. The two long-chain alkyl groups may be the same type or different types from each other, and the two long-chain alkenyl groups may be the same type or different types from each other.

Examples of the quaternary ammonium salt include dioleoyloxytrimethylammonium propane chloride (DOTAP, N-[1-(2,3-dioleoyloxy) propyl]-N,N,N-trimethylammonium chloride), dioctadecenyltrimethylammonium propane chloride (DOTMA, N-[1-(2,3-dioleyloxy) propyl]-N,N,N-trimethylammonium chloride), didecyldimethylammonium chloride, didecyldimethylammonium bromide, dilauryldimethylammonium chloride, dicetyldimethylammonium chloride, dicetyldimethylammonium bromide, distearyldimethylammonium chloride, distearyldimethylammonium bromide, dioleyldimethylammonium chloride, dibehenyldimethylammonium chloride, dibehenyldimethylammonium bromide, dipalmitoylethyl hydroxyethylmonium metosulfate, and distearoylethyl hydroxyethylmonium metosulfate.

### [Liquid Crystal Phase]

It is preferable that the composition for a living body according to the embodiment of the present invention forms a liquid crystal phase through absorption of water or moisture upon contact with water. Because of the formation of the liquid crystal phase, the film formed of the composition for a living body according to the embodiment of the present invention exhibits an excellent elastic modulus and strain resistance.

It is noted that in the present specification, the term “hygroscopic” refers to absorbing moisture. Examples of the moisture include water in the atmosphere and water in the exhalation. In addition, i the present specification, the water absorption refers to the absorption of water (excluding moisture).

The thickness of the film formed of the composition for a living body according to the embodiment of the present invention is not particularly limited, but is, for example, 0.1 μm to 1 mm, and preferably 1 to 100 μm.

Examples of the water to be brought into contact with the composition for a living body according to the embodiment of the present invention include water (moisture) in the atmosphere, water (moisture) in exhalation, pure water, and water contained in an aqueous fluid other than water. Examples of the aqueous fluid other than water include saliva, tissue fluid, blood, and lymph.

In a case of using the composition for a living body according to the embodiment of the present invention, the amount of water to be brought into contact with the composition for a living body according to the embodiment of the present invention is not particularly limited, but is preferably 1,000% by mass or less and more preferably 500% by mass or less with respect to the total mass of the composition for a living body according to the embodiment of the present invention. The lower limit of the amount of the water with respect to the total mass of the composition for a living body according to the embodiment of the present invention, which is used in a case of being brought into contact with the composition for a living body according to the embodiment of the present invention, is not particularly limited, and examples thereof include more than 1% by mass.

A temperature at which the composition for a living body according to the embodiment of the present invention is brought into contact with water is not particularly limited, but is preferably 20° C. to 40° C. and more preferably 35° C. to 40° C.

The liquid crystal phase which can be formed by bringing the composition for a living body according to the embodiment of the present invention into the contact with water is not particularly limited, and is any one selected from the group consisting of an inverse hexagonal columnar (H2) phase (a W/O hexagonal columnar phase), a hexagonal columnar (H1) phase (an O/W hexagonal columnar phase), a lamellar (La) phase, a sponge (V2) phase, a bicontinuous cubic (L3) phase, and a mixed state of two or more thereof in many cases. The liquid crystal phase preferably has an inverse hexagonal columnar (H2) phase or a hexagonal columnar (H1) phase.

Among these, the composition for a living body according to the embodiment of the present invention preferably forms an inverse hexagonal columnar (H2) phase by water absorption or moisture absorption, and more preferably forms an inverse hexagonal columnar (H2) phase by moisture absorption.

In the composition for a living body according to the embodiment of the present invention, the liquid crystal phase may undergo a phase transition by further absorbing water after absorbing moisture. Examples of the above-described phase transition include a phase transition from an inverse hexagonal columnar (H2) phase to a hexagonal columnar (H1) phase. Specifically, as described above, examples thereof include an aspect in which the moisture in a small amount of water (moisture) such as water in the atmosphere and water in exhalation is absorbed to form a liquid crystal phase (for example, an inverse hexagonal columnar (H2) phase), and then a larger amount of water than water (moisture) such as in the atmosphere from the air (moisture), such as water sprayed by spraying, artificial saliva, an aqueous fluid, exudate from a skin, and water ingested is absorbed to cause the above-described liquid crystal phase to undergo a phase transition to another liquid crystal phase (for example, a hexagonal columnar (H1) phase).

### [Method for Producing Composition for Living Body]

As a method for producing the composition for a living body according to the embodiment of the present invention, for example, a method of mixing the compound X, the phospholipid, the compound Y, and optionally an optional component at a predetermined mixing ratio can be used.

The method of mixing is not particularly limited, and a conventionally known method can be used.

### [Application of Composition for Living Body]

The composition for a living body according to the embodiment of the present invention can be used on a living body.

In a living body, the composition for a living body according to the embodiment of the present invention can be used, for example, for the purpose of assisting or repairing a part that does not exhibit an original function due to an injury, a disease, or the like (for example, a site such as a skin, hair, or a mucous membrane, the same applies to “part” hereinafter), and a part where the function is decreased. Among those, the composition for a living body according to the embodiment of the present invention can be preferably used for a skin or a mucous membrane (particularly, for an oral mucous membrane protection and gastrointestinal tract protection).

### [Method for Using Composition for Living Body]

Examples of the method for using the composition for a living body according to the embodiment of the present invention include a method in which the composition for a living body according to the embodiment of the present invention is disposed on a portion having the symptoms described above, and then the composition for a living body according to the embodiment of the present invention is brought into contact with water.

In a case where the composition for a living body according to the embodiment of the present invention is used on the skin, for example, the composition for a living body according to the embodiment of the present invention may be applied onto the skin in the atmosphere, and water or an aqueous solution containing water may be added to the composition for a living body according to the embodiment of the present invention as necessary.

The composition for a living body according to the embodiment of the present invention on the skin can form a film (preferably a liquid crystal film) by being brought into contact with, for example, any of water in the atmosphere, water sprayed by spraying, the above-described aqueous fluid, and exudate from the skin.

In a case where the composition for a living body according to the embodiment of the present invention is used for a mucous membrane, the composition for a living body according to the embodiment of the present invention may be disposed on the mucous membrane, and water or an aqueous solution containing water may be added to the composition for a living body according to the embodiment of the present invention as necessary.

The composition for a living body according to the embodiment of the present invention on the mucous membrane can form a film (preferably a liquid crystal film) by being brought into contact with, for example, any of water in the atmosphere, water in the exhalation, water sprayed by spraying, the above-described aqueous fluid, or water ingested.

In particular, in a case where the composition for a living body according to the embodiment of the present invention is applied to an oral mucous membrane, since the composition for a living body according to the embodiment of the present invention is adhered (applied) to the oral mucous membrane and thus brought into contact with any of water in the atmosphere, water in the exhalation, or water in the saliva to form the film, the handling is simple. In addition, in a case where the amount of saliva is small, it is sufficient to supply water by spraying water or artificial saliva after attaching the composition for a living body according to the embodiment of the present invention to the oral mucous membrane.

### EXAMPLES

Hereinafter, the present invention will be described in more detail based on Examples.

The materials, the amounts of materials used, the proportions, the treatment details, the treatment procedure, and the like shown in Examples below may be modified as appropriate as long as the modifications do not depart from the spirit of the present invention. Therefore, the scope of the present invention should not be construed as being limited to Examples shown below.

### [Evaluation]

### [Preparation of Composition for a Living Body]

The composition for a living body of each of Examples and Comparative Examples were prepared by mixing the following raw materials such that each component shown in Tables 1 and 2 would have the blending amount (parts by mass) shown in the same tables.

In Tables 1 and 2, the details of raw materials of each component are as follows.
Selachyl alcohol (NIKKOL selachyl alcohol V, manufactured by Nikko Chemicals Co., Ltd.): compound X
Phospholipid (LIPOID P100, manufactured by Lipoid GmbH, containing 90% by mass or more of phosphatidylcholine)
Propylene glycol (manufactured by FUJIFILM Wako Pure Chemical Corporation): compound Y
Ethanol (manufactured by FUJIFILM Wako Pure Chemical Corporation): compound Y
1,3-Butylene glycol (manufactured by FUJIFILM Wako Pure Chemical Corporation): compound Y
1-Pentanol (manufactured by FUJIFILM Wako Pure Chemical Corporation)

### [Elastic Modulus]

The elastic modulus of the film formed by the composition for a living body was measured using a rheometer device (MCR302) and a measurement jig PP25 according to the following procedure.

Using a SUS jig with a thickness of 200 μm having a hole with a diameter of 25 mm, the composition for a living body was applied onto a base to form a coating with a diameter of 25 mm and a thickness of 200 μm, and the base was set in a rheometer device. Water was sprayed 3 times (the amount of water sprayed 3 times was 0.3 mL) onto the composition for a living body, which was applied as described above, using a trusco finger spray (TFSB-20), and the composition was allowed to stand for 1 minute.

After 1 minute, the strain sweep measurement was performed in a range of 0.001% to 1,000% at a measurement temperature of 25° C., 50% rh (relative humidity), a gap of 200 μm, a frequency of 1 Hz, and Nf of 1 N. From the value of the storage elastic modulus G′ at a shear strain of 0.1% obtained, the elastic modulus of the film formed of the composition for a living body was evaluated according to the evaluation standard below. It is preferable that the evaluation is B or higher.
A: Storage elastic modulus G′ at a shear strain of 0.1% was 30,000 Pa or more
B: Storage elastic modulus G′ at a shear strain of 0.1% was 1,000 Pa or more and less than 30,000 Pa
C: Storage elastic modulus G′ at a shear strain of 0.1% was less than 1,000 Pa.

### [Strain Resistance]

Regarding the storage elastic modulus G′ and the loss elastic modulus G″ obtained by the strain sweep measurement in the [Elastic modulus] described above, the strain resistance was evaluated according to the evaluation standard below from the shear strain (%) at which G′=G″ (that is, the shear strain at the intersection between the storage elastic modulus G′ and the loss elastic modulus G″ in a graph with shear strain on the horizontal axis and elastic modulus on the vertical axis). The strain resistance is preferably evaluated as C or higher.
A: Shear strain (%) at which G′=G″ was 30% or more.
B: Shear strain (%) at which G′=G″ was 10% or more and less than 30%
C: Shear strain (%) at which G′=G″ was 5% or more and less than 10%
D: Shear strain (%) at which G′=G″ was less than 5%

### [Liquid Viscosity]

The liquid viscosity of the composition for a living body was evaluated using a rheometer device (MCR302) and a measurement jig PP25. The above-described composition for a living body was set on a base of a device, and the liquid viscosity of the composition for a living body was measured under conditions of a measurement temperature of 25° C., 50% rh (relative humidity), a gap of 1 mm, and a shear rate of 0.1 to 1,000 (1/s). The liquid viscosity was evaluated according to the evaluation standard below from the viscosity at a shear rate of 0.1 (1/s).
A: Viscosity at a shear rate of 0.1 (1/s) was less than 10,000 cP.
B: Viscosity at a shear rate of 0.1 (1/s) was 10,000 cP or more and less than 100,000 cP.
C: Viscosity at a shear rate of 0.1 (1/s) was 100,000 cP or more.

### [Film Formation Rate]

Each composition for a living body was applied to a slide glass to a thickness of 200 μm under the conditions of 25° C. and 50% rh (relative humidity).

After the application, the composition for a living body on the slide glass was observed with a polarizing microscope after 1 minute, 30 minutes, 1 hour, and 24 hours. In the above observation, a time required for the composition for a living body on the slide glass to form a liquid crystal phase (time required for the field of view of the polarizing microscope to change from a dark field to a bright field) was recorded. In a case where the composition for a living body forms a liquid crystal phase, a field of view of a polarizing microscope is a bright field.

From the above observation, the film formation rate was evaluated from the time until the liquid crystal phase was formed.
A: A liquid crystal phase was formed in 1 minute or less.
B: A liquid crystal phase was formed in more than 1 minute and 1 hour or less.
C: A liquid crystal phase was formed in more than 1 hour and less than 24 hours.
D: A liquid crystal phase was not formed even after 24 hours.

### [Liquid Crystal Phase]

Each of the compositions for a living body was applied onto a slide glass to have a thickness of 200 μm, and small-angle X-ray scattering (SAXS) measurement was performed using a sample in which the above-described slide glass had been allowed to stand at 25° C. and 50% rh (relative humidity) for 24 hours or more, and the liquid crystal structure was determined. For the SAXS measurement, a small-angle X-ray scattering measuring device Nanopix (Cu Ka, 40 kV/30 mA) manufactured by Rigaku Corporation was used. The results of the SAXS measurement are shown by a scattering curve using a scattering intensity as the ordinate and a scattering vector length (q/nm⁻¹) as the abscissa. For each peak on the obtained scattering curve, a ratio of the scattering vector length (q/nm⁻¹) was measured to identify the liquid crystal structure.

In a case where at least three peaks were observed on the scattering curve and the ratio of the scattering vector lengths of the peaks was about 1:√3:√4, it was determined that the liquid crystal phase was an inverse hexagonal columnar phase (H2). The ratio of the scattering vector lengths of the three peaks of 1:√3:√4 is specific to the inverse hexagonal columnar phase (H2).

### [Result]

Hereinafter, Tables 1 and 2 show the composition and the evaluation results of each composition for a living body.

In the tables, in the evaluation column, “N.D.” was used in a case where the above-described evaluation could not be performed because of the properties of the composition.

In the evaluation of the liquid crystal phase, “H2” was used in a case where the liquid crystal phase was an inverse hexagonal columnar phase, and “N.D.” was used in a case where no liquid crystal phase was formed.

TABLE 1

| | | Example | Example | Example | Example | Example | Example | Example |
|---|---|---|---|---|---|---|---|---|
| | | 1 | 2 | 3 | 4 | 5 | 6 | 7 |
| Composition | Selachyl alcohol | 40 | 45 | 50 | 60 | 70 | 80 | 90 |
| for living | Phospholipid | 60 | 55 | 50 | 40 | 30 | 20 | 10 |
| body | Propylene glycol | 5 | 5 | 5 | 5 | 5 | 5 | 5 |
| | Ethanol | | | | | | | |
| | 1,3-Butylene | | | | | | | |
| | glycol | | | | | | | |
| | 1-Pentanol | | | | | | | |
| Evaluation | Elastic modulus | A | A | A | A | A | A | B |
| | Strain resistance | B | B | B | A | A | B | B |
| | Liquid viscosity | C | C | B | A | A | A | A |
| | Film formation | A | A | A | A | B | C | C |
| | rate | | | | | | | |
| | Liquid crystal | H2 | H2 | H2 | H2 | H2 | H2 | H2 |
| | phase | | | | | | | |

TABLE 2

| | | Example | Example | Example | Comparative | Comparative | Comparative | Comparative |
|---|---|---|---|---|---|---|---|---|
| | | 8 | 9 | 10 | Example 1 | Example 2 | Example 3 | Example 4 |
| Composition | Selachyl | 60 | 60 | 60 | 60 | 100 | 60 | 60 |
| for living | alcohol | | | | | | | |
| body | Phospholipid | 40 | 40 | 40 | 40 | 0 | 40 | 40 |
| | Propylene | 10 | | | 20 | 5 | 0 | |
| | glycol | | | | | | | |
| | Ethanol | | 5 | | | | | |
| | 1,3-Butylene | | | 5 | | | | |
| | glycol | | | | | | | |
| | 1-Pentanol | | | | | | | 5 |
| Evaluation | Elastic | A | A | A | A | C | N.D. | N.D. |
| | modulus | | | | | | | |
| | Strain | B | A | A | D | C | N.D. | N.D. |
| | resistance | | | | | | | |
| | Liquid | A | A | A | A | A | N.D. | N.D. |
| | viscosity | | | | | | | |
| | Film | B | A | A | C | D | D | D |
| | formation | | | | | | | |
| | rate | | | | | | | |
| | Liquid | H2 | H2 | H2 | H2 | N.D. | N.D. | N.D. |
| | crystal | | | | | | | |
| | phase | | | | | | | |

In the composition of Comparative Example 1 in which the content of the compound Y was more than 10% by mass with respect to the total mass of the compound X and the phospholipid, the strain resistance did not satisfy the desired level.

In the composition of Comparative Example 2, which did not contain a phospholipid, a film could not be formed, and the elastic modulus and the strain resistance did not satisfy the desired level.

In the composition of Comparative Example 3, in which the content of the compound Y was less than 1% by mass with respect to the total mass of the compound X and the phospholipid, the composition was not uniform, a film could not be formed, any evaluation could not be performed, and the desired properties were not exhibited.

In the composition of Comparative Example 4, in which an alcohol having 5 or more carbon atoms was used, the components thereof were not dissolved, a film could not be formed, any evaluation could not be performed, and the desired properties were not exhibited.

In contrast, it has been confirmed that the composition for a living body according to the embodiment of the present invention, a film could be formed upon contact with water and the elastic modulus and strain resistance of the formed film was excellent.

From the comparison of Examples 1 to 7, it was confirmed that in a case where the mass ratio of the content of the compound X to the content of the phospholipid was 70/30 to 50/50, the liquid viscosity and the film formation rate were more excellent, and in a case where the mass ratio was 65/35 to 55/45, the effect of the present invention was more excellent.

From the comparison of Examples 1 to 7, it was confirmed that in a case where the content of the compound X is 45% by mass or more with respect to the total mass of the composition for a living body, the liquid viscosity is more excellent, and in a case where the content of the compound X is 50% by mass or more, the strain resistance and the liquid viscosity are further excellent. In addition, it was confirmed that in a case where the content of the compound X is 80% by mass or less with respect to the total mass of the composition for a living body, the modulus of elasticity is more excellent, in a case where the content thereof is 70% by mass or less, the strain resistance and the film formation rate are further excellent, and in a case where the content thereof is 65% by mass or less, the film formation rate is particularly excellent.

From the comparison between Example 4 and Example 8, it was confirmed that in a case where the content of the compound Y was 3% to 8% by mass with respect to the total mass of the compound X and the phospholipid, the strain resistance and the film formation rate were more excellent.

What is claimed is:

1. A composition for a living body, comprising:
a compound X selected from the group consisting of an α-monoalkyl glyceryl ether and an α-monoalkenyl glyceryl ether;
a phospholipid; and
a compound Y selected from the group consisting of an alcohol having 4 or less carbon atoms and a polyalkylene oxide,
wherein a content of the compound Y is 1% to 10% by mass with respect to a total mass of the compound X and the phospholipid.

2. The composition for a living body according to claim 1,
wherein the compound X is selected from the group consisting of selachyl alcohol, batyl alcohol, and chimyl alcohol.

3. The composition for a living body according to claim 1,
wherein the compound X is selachyl alcohol.

4. The composition for a living body according to claim 1,
wherein a mass ratio of a content of the compound X to a content of the phospholipid is 70/30 to 50/50.

5. The composition for a living body according to claim 1,
wherein a content of water is 0% to 10% by mass with respect to a total mass of the composition for a living body.

6. The composition for a living body according to claim 1,
wherein a content of the compound X is 50% by mass or more with respect to a total mass of the composition for a living body.

7. The composition for a living body according to claim 1,
wherein the composition for a living body forms an inverse hexagonal columnar phase through absorption of water or moisture.

8. The composition for a living body according to claim 1,
wherein the composition for a living body is for a skin or a mucous membrane.

9. The composition for a living body according to claim 2,
wherein the compound X is selachyl alcohol.

10. The composition for a living body according to claim 2,
wherein a mass ratio of a content of the compound X to a content of the phospholipid is 70/30 to 50/50.

11. The composition for a living body according to claim 2,
wherein a content of water is 0% to 10% by mass with respect to a total mass of the composition for a living body.

12. The composition for a living body according to claim 2,
wherein a content of the compound X is 50% by mass or more with respect to a total mass of the composition for a living body.

13. The composition for a living body according to claim 2,
wherein the composition for a living body forms an inverse hexagonal columnar phase through absorption of water or moisture.

14. The composition for a living body according to claim 2,
wherein the composition for a living body is for a skin or a mucous membrane.

15. The composition for a living body according to claim 3,
wherein a mass ratio of a content of the compound X to a content of the phospholipid is 70/30 to 50/50.

16. The composition for a living body according to claim 3,
wherein a content of water is 0% to 10% by mass with respect to a total mass of the composition for a living body.

17. The composition for a living body according to claim 3,
wherein a content of the compound X is 50% by mass or more with respect to a total mass of the composition for a living body.

18. The composition for a living body according to claim 3,
wherein the composition for a living body forms an inverse hexagonal columnar phase through absorption of water or moisture.

19. The composition for a living body according to claim 3,
wherein the composition for a living body is for a skin or a mucous membrane.

20. The composition for a living body according to claim 4,
wherein a content of water is 0% to 10% by mass with respect to a total mass of the composition for a living body.
